(19) United States
(12) Patent Application Publication
(10) Pub. No.: US 2021/0251814 A1
(43) Pub. Date: Aug. 19, 2021

(54) SHAPED PERMEABLE MATERIAL COMPOSITE FOR USE IN AN ABSORBENT ARTICLE

(71) Applicant: Essity Hygiene and Health Aktiebolag, Goteborg (SE)

(72) Inventors: Asa Jönegren, Goteborg (SE); Oscar Lipschutz, Goteborg (SE); Natalia Maria Petro Bedoya, Goteborg (SE)

(21) Appl. No.: 16/973,129

(22) PCT Filed: Jun. 20, 2018
(86) PCT No.: PCT/SE2018/050671

§ 371 Date: Dec. 8, 2020

(51) Int. Cl. A61F 13/15; A61F 13/531; A61F 13/84

(52) U.S. Cl. CPC A61F 13/15804; A61F 13/15723; A61F 2013/530481; A61F 13/531; A61F 13/8405; A61F 13/15739

(57) ABSTRACT

A permeable material composite is provided for use in an absorbent article. The permeable material composite includes a permeable material layer that is a low-strength material layer and having a tensile strength of less than 3 N/mm. The permeable material layer has an edge having a contour and being a shaped edge providing the permeable material composite with a non-rectangular shape. The permeable material layer is reinforced along the shaped edge with a reinforcement material that is attached to the permeable material layer. The reinforcement material has a shaped outer edge having a contour that coincides fully or partly with the contour of the shaped edge of the permeable material layer.

### CROSS-REFERENCE TO RELATED APPLICATION

This application is a national phase entry of, and claims priority to, International Application No. PCT/SE2018/050671, filed Jun. 20, 2018. The above-mentioned patent application is incorporated herein by reference in its entirety.

### TECHNICAL FIELD

This application is related to a shaped permeable material composite having reinforced shaped edges. This application is also related to a method for producing the shaped permeable material composite.

### BACKGROUND

Absorbent articles such as diapers, sanitary pads, and incontinence pads may employ permeable material layers having a relatively low tensile strength, which may be desired for numerous reasons. For example, such permeable material layers may advantageously form part of a liquid receiving body in an absorbent article, e.g. be used between the topsheet and the absorbent core for acquisition and distribution of liquid and/or in an absorbent core. Thin material layers may be used for providing thinner absorbent article to wear and fit better under clothing, they are also more compact in the package, making the diapers easier for the consumer to carry and store. Compactness in packaging also results in reduced distribution costs for the manufacturer and distributor.

Material layers for use in absorbent articles may be cut in shaped configurations, such as, for example, hourglass shapes or triangular shapes. For shaped configurations a certain amount of waste is created. Such waste will increase the cost to manufacture the absorbent article. Therefore, it is desirable to minimize the trim material width to reduce the material waste.

The production of absorbent articles are made in high-speed production systems, interruptions in such production lines are undesired. When trimming weak material layers into shaped configurations frequent interruption due to breakage of trim material during removal of the material may create an inefficient production and defective products, especially when the trim material width is minimized to reduce waste.

Therefore, it would be desirable to offer improvements in the method for producing a shaped permeable material layer and reduce the number of defective products resulting from the production.

### SUMMARY

In order to achieve these technical objectives, this application provides, in a first embodiment, a permeable material composite for use in an absorbent article. The permeable material composite comprises a permeable material layer being a low-strength material layer and having a tensile strength of less than 3 N/mm. The permeable material layer has an edge, the edge having a contour and being a shaped edge providing the permeable material composite with a non-rectangular shape. The permeable material layer is reinforced along the shaped edge with a reinforcement material being attached to the permeable material layer. The reinforcement material has a shaped outer edge having a contour and the contour of the shaped outer edge of the reinforcement material coincides fully or partly with the contour of the shaped edge of the permeable material layer.

The edge of the permeable material and the outer edge of the reinforcement material may coincide with the edge of the permeable material composite.

The permeable material layer may have at least two shaped edges, at least three shaped edges or four shaped edges and thus be shaped, such as cut or severed, around the complete perimeter of the permeable material layer to have a shape other than a rectangular shape, giving also the permeable material composite a non-rectangular shape. The shape may for example be an hourglass shape, a round shape or an oval shape. The reinforcement material may, fully or partly, extend along at least the shaped edge and have an outer edge having a contour coinciding with the shaped edge. If the permeable material layer has more than one shaped edge, the reinforcement material may extend along some or each of the shaped edges of the permeable material layer. If the permeable material layer has two or more shaped edges, the reinforcement material may have two or more outer edges and extend along the two or more shaped edges of the permeable material layer and wherein the outer edges of the reinforcement material may coincide with two or more shaped edges of the permeable material.

The permeable material layer may have a length in a longitudinal direction extending between a first and a second transverse edge and a width extending between a first and a second transverse edge. At least one of the longitudinal or transverse edges, may be a shaped edge having a contour. The longitudinal and transverse edges may be connected by rounded corner portions.

The term “absorbent article” refers to products that are placed against the skin of the wearer to absorb and contain body exudates, like urine, feces and menstrual fluid such as for example a sanitary pad, a panty liner, an incontinence pad or a diaper. This application furthermore refers to disposable absorbent articles, which means articles that are not intended to be laundered or otherwise restored or reused as an absorbent article after use.

The “permeable material composite” may form part of a liquid receiving body, e.g. as an acquisition layer and/or as an absorbent core.

By “low strength permeable material” herein is meant a permeable material layer, such as a liquid permeable layer, having a tensile strength of less than 3 N/mm, the tensile strength being measured in the machine direction of the material during production of the absorbent article. Hence, the thickness and the basis weight of the material may influence whether the material is defined as a low strength permeable material. During cutting and shaping of a permeable material layer, waste material in the form of trim material is removed from the material layer by pulling the trim material with a pulling force leaving the shaped permeable material layer. The definition of the material relates to its ability to remain intact during trimming and removal of the trim material by pulling the trim material with a pulling force during the shaping and cutting step. The pulling force, in a pulling direction, may be 1 N or more.

The tensile strength is measured according to the EDANA method: “Standard Procedure: NWSP 110.4.R0 (15) “Breaking Force and Elongation of Nonwoven Material (Strip Method).” The specimen chosen for testing is Option B. The style of tensile testing machine is Constant-rate of-extension (CRE). The machine constant rate is of 100 mm/min and Max Load N/50 mm in the machine direction, the value is thereafter being recalculated to N/mm. The samples are tested in dry condition.

In absorbent articles layers included therein may be shaped, for example to better fit user anatomy and to correspond to the outer contour of the absorbent article. Low-strength permeable material layers used in absorbent articles to provide for example greater flexibility, breathability and absorbency may be difficult to shape and the cut edges may break and cause irregularities along the outer edges. For pre-treated materials, such as pre-treatment with active substances, or fibrous materials mixed with particles, such as for example superabsorbent particles, leakage along shaped edge portion may also increase.

It has been found that when reinforcing low-strength permeable material layers along the one or more shaped edge(s) with a reinforcement material prior to shaping of the layer irregularities and breaks which otherwise may occur along the shaped edge(s) during a cutting step and removal of the trim material may be avoided.

In one embodiment, the permeable material layer is a liquid permeable fibrous layer or a liquid permeable foam layer. The permeable material layer may be a liquid absorbent layer. The foam layer may be an open-cell polymeric foam layer.

The foams are open-celled polymeric foams. A foam material is “open-celled” if at least about 80% of the cells in the foam structure that are at least 1 μm size are in liquid communication with at least one adjacent cell.

In another embodiment, the fibers in the liquid permeable fibrous layer consist of non-absorbent synthetic fibers, such as thermoplastic polymeric fibers, for example polyolefin or polyester fibers. The liquid permeable fibrous layer may also be an airlaid fibrous layer comprising absorbent fibers, such as cellulose fibers and synthetic fibers, such as rayon fibers, polyolefin fibers and polyester fibers.

In a further embodiment, the permeable material layer comprises superabsorbent particles.

The fact that one or more of the shaped longitudinal and/or transverse edges are reinforced with a reinforcing material prevent, or at least reduces, losses of the superabsorbent particles both during manufacturing and when used in an absorbent article. Superabsorbent particles mixed within a permeable material layer, such as for example a fibrous layer may otherwise during manufacturing and also when used in an absorbent article leak from shaped edge portions for low-strength fibrous material layers.

In yet another embodiment, the reinforcing material is a tissue material or a nonwoven material, such as a spunbond nonwoven material, such as a nonwoven material or a tissue material having a basis weight of from 16 to 22 gsm. The width of the tissue material or the nonwoven material be 25 mm or greater.

It has been found that using a tissue material or a nonwoven material with a basis weight of from 16 to 22 gsm as reinforcing material as disclosed herein may reduce the risk with irregular edges caused by breaks and ruptures for low-strength shaped materials. A width of the tissue material or of the nonwoven material is advantageously greater than 25 mm for providing an enhanced reinforcement to the low-strength shaped material.

In one embodiment, the tensile strength of the permeable material composite is higher than the tensile strength of the permeable material layer alone.

In another embodiment, the tensile strength of the reinforcing material is 0.1 N/mm or more. Optionally, the reinforcing material has a tensile strength greater than the tensile strength of the permeable material layer.

In yet another embodiment, the permeable material layer has a tensile strength of from 0.05 to 3 N/mm.

In a further embodiment, the shaped edge(s) of the permeable material layer is/are co-shaped with the outer shaped edge(s) of the reinforcing material.

In one embodiment, the first and the second longitudinal edges are shaped first and second longitudinal edges.

In another embodiment, the reinforcing material further comprises an active ingredient, such as an odor control substance, a print, a hydrophobic composition and/or elastic elements.

In accordance with a second embodiment, an absorbent article includes a liquid permeable topsheet and a backsheet, wherein the permeable material composite according to the first embodiment is enclosed between the liquid permeable topsheet and the backsheet.

The liquid permeable topsheet can be any suitable topsheet material as known by the person skilled in the art and may be fibrous topsheet material composed of a nonwoven material, e.g. spunbonded, meltblown, carded, hydroentangled, wet-laid etc. Suitable nonwoven materials can be composed of natural fibers, such as wood pulp or cotton fibres, synthetic thermoplastic fibres, such as polyolefins, polyesters, polyamides and blends and combinations thereof or from a mixture of natural and synthetic fibres. Further examples of topsheet materials are porous foams. The materials suited as topsheet materials should be soft and non-irritating to the skin and be readily penetrated by body fluid, such as urine or menstrual fluid. The topsheet material may essentially be constituted of non-absorbent fibers, such as synthetic thermoplastic fibers, such as such as polyolefins, polyesters, polyamides and blends and combinations thereof. The synthetic fibers may be monocomponent fibers, bicomponent fibers or multicomponent fibers including polyesters, polyamides and/or polyolefins such as polypropylene and polyethylene.

The backsheet may consist of a thin plastic film, e.g. a polyethylene or polypropylene film, a nonwoven material coated with a liquid impervious material, a hydrophobic nonwoven material, which resists liquid penetration. Laminates of plastic films and nonwoven materials may also be used. The backsheet material can be breathable so as to allow vapor to escape from the absorbent structure, while still preventing liquids from passing through the backsheet material.

In accordance with a third embodiment, a method is provided for producing a permeable material composite having one or more shaped reinforced edges for use in an absorbent article. The method includes the steps of:
a) providing a web of a permeable material having a tensile strength of less than 1 N/mm;
b) providing a web of a reinforcing material;
c) applying the reinforcing material web over the permeable material web and attaching the reinforcing material web to the permeable material web to provide a permeable material composite web comprising a reinforced overlap region; and
d) forming the permeable material composite having one or more shaped reinforced edge(s) by cutting or severing along a cutting line at least partly arranged within the overlap region and removing trim material from the permeable material composite.

In one embodiment, the trim material may be removed by pulling the trim material with a pulling force, the pulling force may be 1 N or higher.

Step d) may also including forming the permeable material composite having two, three or four shaped reinforced edges by cutting or severing along a cutting line at least partly arranged within the overlap region and removing trim material from the permeable material composite.

After cutting or severing of a material (web) during shaping of the material, trim material remaining may be removed from the shaped material (web) by pulling the trim material with a pulling force. Due to the low strength of the permeable material web according to the present disclosure, the trim material remaining after cutting operations often breaks when being removed from the permeable material web, which causes machine interruptions. It may additionally result in irregularities along the shaped edge(s) of the low-strength permeable material. Interruptions in machines and defect product are very costly.

It has been found that when reinforcing the low-strength permeable material layer according the present disclosure over the region(s) where the web is to be cut and by reinforcing the material becoming trim material after the cutting step, the otherwise frequent machine stops due to breakage of trim material may be significantly reduced.

The reinforcing material may at least be attached, continuously or discontinuously, to the first web of permeable material over and on each side of the intended cutting line, i.e. on the side forming the shaped permeable material layer and on the side constituting the trim material after the cutting step.

The web of permeable material may have longitudinal edge portions extending in a longitudinal direction and step b) may involve conveying the permeable material web in a machine direction corresponding to the longitudinal direction of the web of permeable material.

In another embodiment, in step c), the reinforcing material is applied to overlap one or both of the first and the second longitudinal edge portions of the web of permeable material forming overlap regions along the one or both of the first and the second longitudinal edge portions. Step d) may include cutting at least partly within the overlap region(s) and removing trim material from the permeable material composite web forming one or two shaped reinforced longitudinal edges. Optionally and in a subsequent step, the shaped permeable material composite web may be cut with transverse cuts forming individual permeable material composites. The shape of the transverse cuts and the intended final shape of the permeable material composites transverse edges may be such that no trim material remains. In other words, that the shape of the rear edge of a permeable material composite conforms with the front edge of an adjacent permeable material composite at a common borderline, as seen in the longitudinal direction LD of the permeable material composite web, such as for example with a straight cut.

Alternatively, smaller trim material pieces or fragments may be formed with the transverse cuts, which for example may be removed by vacuum.

Shaping by cutting or severing the longitudinal edge portions of the web of permeable material may be the most critical cutting step as it is desired to cut with as low material waste as possible, i.e. by using webs of permeable material with a width as close as possible to the width or length, depending on the orientation if the final cut layer during manufacturing.

In one embodiment, the tensile strength of the permeable material composite is higher than the tensile strength of the web of permeable material alone.

In a further embodiment, the reinforcing material has a tensile strength greater than the tensile strength of the web of a permeable material.

The fact that the reinforcing material has a tensile strength which is greater than the tensile strength of the permeable material provides the reinforced overlap region with a higher average strength increasing the integrity of the web in the region where the permeable material layer is cut, thereby reduces the number of machine interruptions caused by trim breakage and the number of defect products.

In another embodiment, the one or more trim material each has a width and a minimum width of the trim material is 20 mm or less, such as 12 mm or less, such as 7.5 mm or less. The width of the trim material will vary with the width or length of the shaped permeable material composite and may therefore range from, for example 130 mm to 20 mm, such as between from 100 mm to 7.5 mm.

In yet another embodiment, step c) involves attaching the reinforcing material web to the permeable material web by gluing or by ultrasound.

In a further embodiment, step d) involves cutting the permeable material composite web in a non-rectangular shape.

In one embodiment, the method further comprises the step of:
d) integrating the permeable material composite between a topsheet and a backsheet forming an absorbent article.

In accordance with a fourth embodiment, a method is provided for removing trim material when manufacturing a permeable material composite for use in an absorbent article comprising a topsheet, a backsheet and the permeable material composite therebetween. The method includes the steps of:
a) providing a web of permeable material in a machine direction (MD), said web of permeable material having a tensile strength, as measured in the machine direction;
b) applying a web of reinforcing material over the permeable material web and attaching the reinforcing material web to the permeable material web thereby forming a permeable material composite web;
c) cutting, or severing, the permeable material composite web in the machine direction (MD) so as to form a trim material, the trim material comprising at least a portion of the permeable material web and a portion of the reinforcing material web; and
d) removing the trim material by pulling the trim material with a pulling force, wherein the pulling force is higher than the tensile strength per millimetre of the web of permeable material.

In one embodiment, the permeable material composite web has a higher tensile strength than the pulling force in the step d) of the method according to the fourth embodiment.

In another embodiment, pulling force in step d) is 1 N or more.

In a further embodiment, the web of permeable material has a tensile strength of less than 3 N/mm.

The tensile strength of the material webs should be measured in the intended machine direction.

### BRIEF DESCRIPTION OF THE DRAWINGS

The features and advantages of the invention will be appreciated upon reference to the following drawings. The accompanying drawings, which are incorporated in and constitute a part of this specification, illustrate one or more embodiments of the invention and, together with the general description given above and the detailed description given below, explain the one or more embodiments of the invention.

FIG. 1a is a perspective view showing a method for producing a shaped permeable material composite.

FIG. 1b is a perspective view showing further features of a method for producing a shaped permeable material composite.

FIG. 2a is a perspective view of the application of a reinforcing material over a permeable material web.

FIG. 2b is a perspective view of the application of reinforcing material strips over the longitudinal edge portions of a permeable material web.

FIG. 3 is a top perspective view of a permeable material composite according to one embodiment of this application.

FIG. 4 is a top perspective view of a permeable material composite according to another embodiment of this application.

### DETAILED DESCRIPTION

It is to be understood that the drawings are generally schematic illustrations and that individual components, such as layers of material are not necessarily drawn to scale.

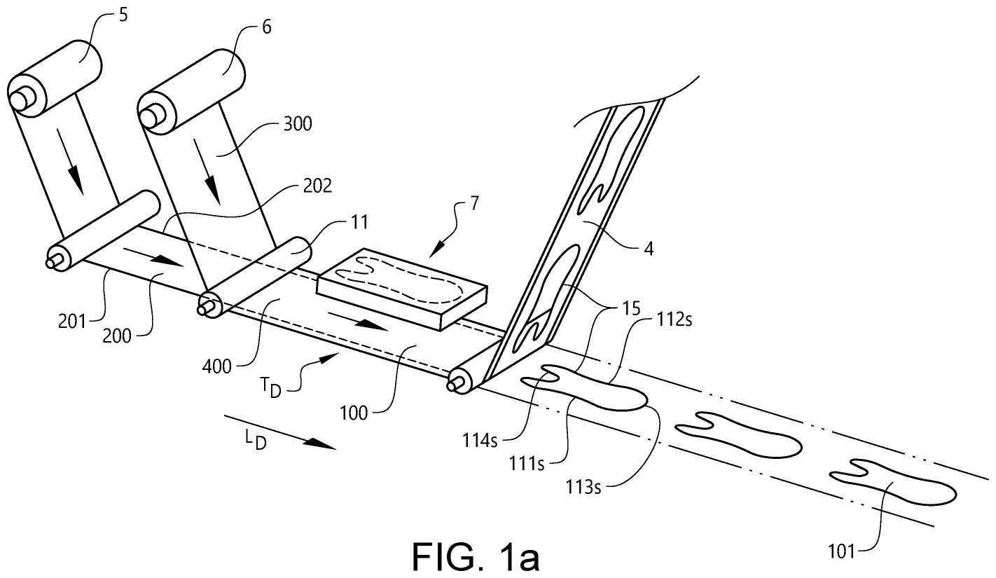

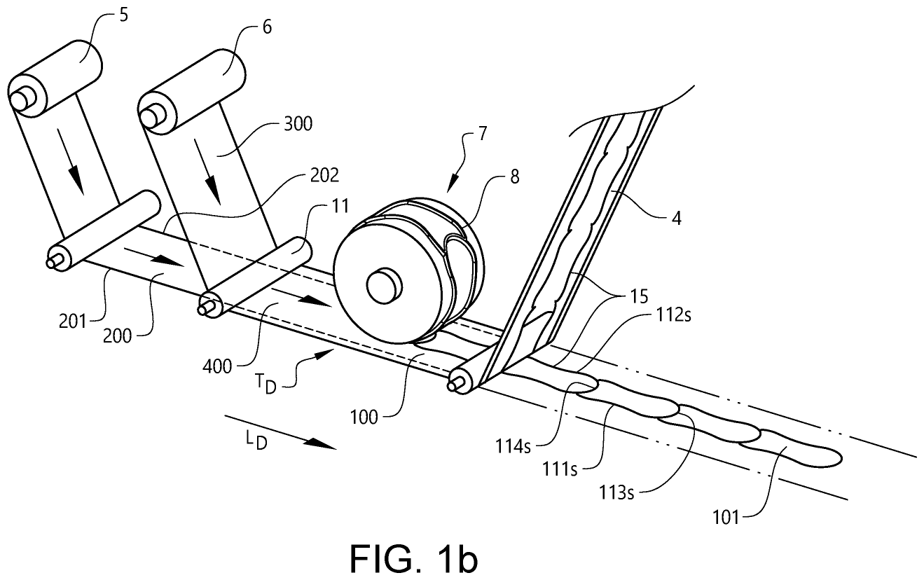

With reference to FIG. 1a, a method of producing a shaped permeable material composite 101 is schematically shown. The permeable material composite 101 is intended to be used in an absorbent article. The method involves providing a first web of a permeable material 100 having a tensile strength of less than 3 N/mm. The first web of a permeable material 200 has first and second longitudinal edge portions 201,202 extending in a longitudinal direction LD and a width extending in a transverse direction TD, being perpendicular to the longitudinal direction LD. The first web of a permeable material 200 is fed in the machine direction indicated by the associated arrows. In addition, a second web of a second material, which is a reinforcing material 300, is provided into the process and is fed in the machine direction indicated by the associated arrows. As shown in FIG. 1a, the first web of permeable material 200 and the second web of reinforcing material 300 may be provided into the process by being unwound from a first supply roll 5 and a second supply roll 6, respectively. However, instead of being unwound from a supply roll, the first web of permeable material 200 and/or the second web of reinforcing material 300 may be provided directly from a respective forming apparatus into the process. The first and the second materials 200,300 will be further described below.

The first web of permeable material 200 and the second web of reinforcing material 300 are attached to each other in a bonding station so as to form a permeable material composite web 100 and reinforced overlap region 400 on the first web of permeable material 200. The overlap region 400 may cover the first web of permeable material 200 completely, as illustrated in FIG. 1a. However, the second web of reinforcing material 300 may also be applied only to parts of the first web of permeable material 200 such as to form an overlap region 400 only partly covering the first web of permeable material 200. The overlap region 400 is at least arranged on the first web of permeable material 200 over the area in which a cutting line 15 is intended to be formed.

The bonding station for attaching the second web of reinforcing material 300 to the first web of permeable material 200 may include any suitable device for attaching two webs of material according to the present disclosure to each other, such as, for example, by glue/adhesive bonding, heat bonding or ultrasonic bonding. The adhesive attachment may be performed by any suitable device for application of adhesive, such as, for example, a slotted glue head or other spraying means.

Ultrasonic bonding may be performed using a rotary ultrasonic horn. Thermal bonding may be conducted by passing the first and the second webs 200,300 between two heated rollers. The heated rollers may have smooth surfaces causing lamination over the whole width of the webs 200,300 or may have pins thereon which form intermittent point bonds between the first and the second web 200,300. The attachment of the reinforcing material 300 to the first web of permeable material 200 may at least be provided over and on each side of the intended cutting line 15.

In the process shown in FIG. 1a, the attachment station is constituted by a rotating lamination roll 11. The first web 200 is fed under the lamination roll 11. For example, the attachment station may alternatively comprise a stationary bar or rod.

Finally, individual permeable material composites 101 having shaped reinforced longitudinal and transverse edges 111s,112s,113s,114s are cut from the permeable material composite web 100 at a cutting station 7. The permeable material composite web 100 is thus cut along the cutting line 15 within the overlap region 400 and trim material 4 is removed by pulling the trim material with a pulling force being higher than the tensile strength per millimetre of the permeable material web 200 but lower than the tensile strength per millimetre of the permeable material composite web 100, as measured in an overlap region 400.

The cutting station 7 may have the form of a cutting roll that includes one or more cutting knives or blades corresponding to the contour of the shaped permeable material composite 101 to be cut from the permeable material composite web 100. The cutting roll may include one or more pairs of obliquely extending pairs of cutting edges or blades and a counter pressure or anvil roll.

FIG. 1b also illustrates a method of producing a permeable material composite 101 according to the present disclosure. The permeable material composite 101 is intended to be included as a component in an absorbent article (not shown) between a topsheet and a backsheet.

The method involves providing a first web of a permeable material 200 having a tensile strength of less than 3 N/mm. The first web of a permeable material 200 has first and second longitudinal edge portions 201,202 extending in a longitudinal direction LD and a width extending in a transverse direction TD, being perpendicular to the longitudinal direction LD. The first web of a permeable material 200 is fed in the machine direction indicated by the associated arrows. A second web 300 of a second material, which is a reinforcing material, is provided into the process and is fed in the machine direction indicated by the associated arrows. As shown in FIG. 1b, the first web of permeable material 200 and the second web of reinforcing material 300 is provided into the process by being unwound from a first supply roll 5 and a second supply roll 6, respectively.

The first web of permeable material 200 and the second web of reinforcing material 300 are attached to each other in a bonding station so as to form a permeable material composite 100 and reinforced overlap region 400 on the first web of permeable material 200. In FIG. 1b the overlap region 400 covers the first web of permeable material 200 completely.

The individual permeable material composites 101 are cut from the permeable material composite web 100 at a cutting station 7 along the cutting line 15 within the overlap region 400 and trim material 4 is removed by pulling the trim material with a pulling force.

The cutting station 7 disclosed in FIG. 1b have the form of a cutting roll including cutting tools such as knives or blades corresponding to the contour of the shaped permeable material composite 101 to be cut from the permeable material composite web 100. As shown in FIG. 1b, there will only be trim material 4 along the longitudinal edge portions of the permeable material composite web 100 as the shape of each cutting tool conforms with the shape of the adjacent cutting tool at a common borderline, as seen in the machine direction. In other words, the shape of a rear edge of a permeable material composite 101 conforms with the front edge of an adjacent permeable material composite 101 at a common borderline, as seen in the longitudinal direction LD of the permeable material composite web 100.

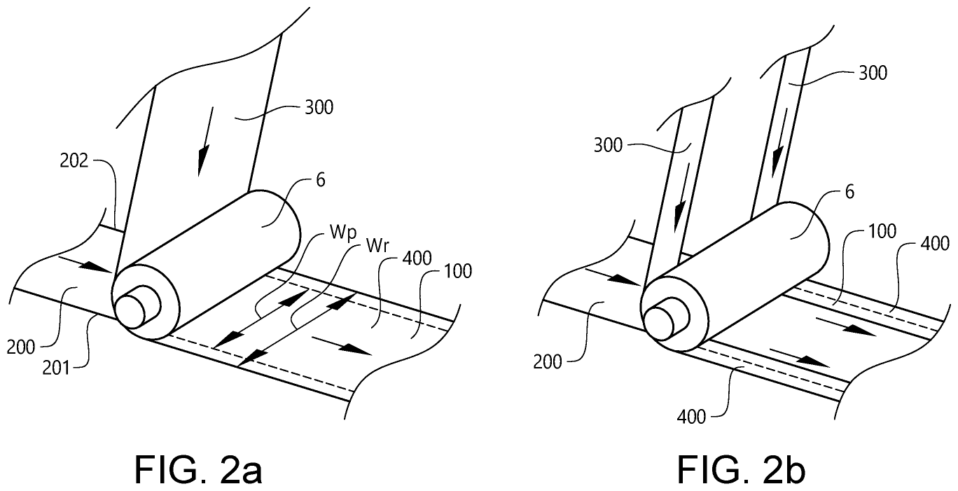

FIG. 2a illustrates one alternative step of applying and attaching the web of reinforcing material 300 over the permeable material web 200 wherein a width wᵣ of the reinforcing material 300 is slightly greater than a width wₚ of the permeable material web 200, such as from 2 to 20 mm wider at each side of the longitudinal edge portions 201,202 of the permeable material web 200.

FIG. 2b illustrates another alternative application of the web of reinforcing material 300 in the form of two strips of reinforcing material over the longitudinal edge portions 201,202 of the permeable material web 200.

After the provision of the web of reinforcing material 300 to the permeable material web 200 in each of the FIGS. 2a and 2b the permeable material composite webs 100 formed may be cut within the overlapping region 400 with a subsequent removal of trim material 4 (see FIGS. 1a and 1b).

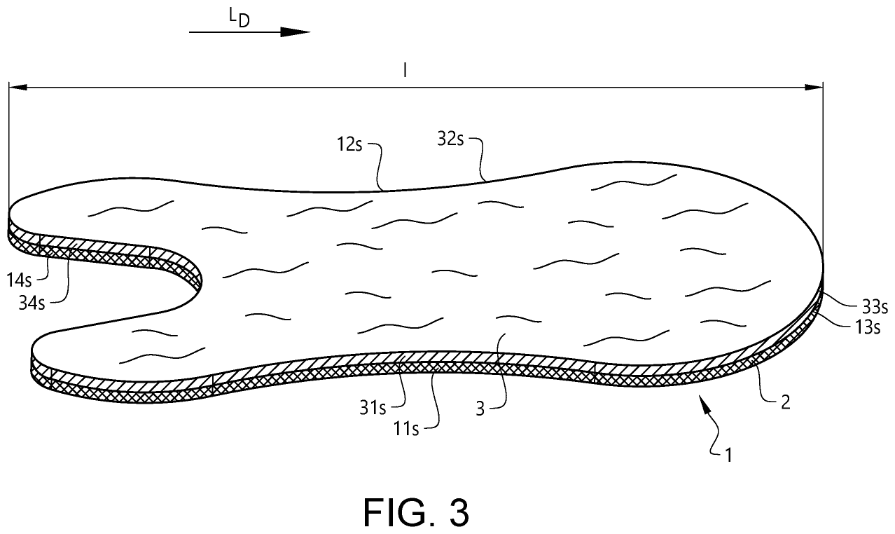

FIG. 3 illustrates a permeable material composite 1 as disclosed herein and for use in an absorbent article. The permeable material composite 1 has a length 1 in a longitudinal direction LD extending between a first shaped transverse edge 13s and a second shaped transverse edge 14s of the permeable material layer 2, which correspond to the transverse edges of the permeable material composite 1. The permeable material composite 1 has a width in a transverse direction, perpendicular to the longitudinal direction LD, extending between a first shaped longitudinal edge 11s and a second shaped longitudinal edge 12s of the permeable material layer 2, which correspond to the longitudinal edges of the permeable material composite 1. The front transverse edges 13s of the permeable material layer and 33s of the reinforcing material 3 are rounded outwardly in a convex shape. The rear transverse edges 14s of the permeable material layer 2 of the reinforcing material 3 each has the shape of two legs extending in a rearwards direction. The first and second longitudinal edges 11s,12s of the permeable material layer 2 and the first and second longitudinal edges 31s,32s of the reinforcing material 3, each has a slightly curved shape, such that the permeable material composite 1 has a slightly inwardly curved waist portion. The permeable material layer 2 has a reinforcing material 3 attached thereto having the same outer contour as the permeable material layer 2. The reinforcing material 3 thus has shaped outer edges 31s,32s,33s,34s with contours coinciding fully with the contours of the edges 11s,12s,13s,14s of the permeable material layer 1.

The permeable material layer 2 is a fibrous layer comprising non-absorbent synthetic fibers, the layer has a tensile strength of less than 3 N/mm. The reinforcing layer 3 is a nonwoven layer completely covering the permeable material layer 2.

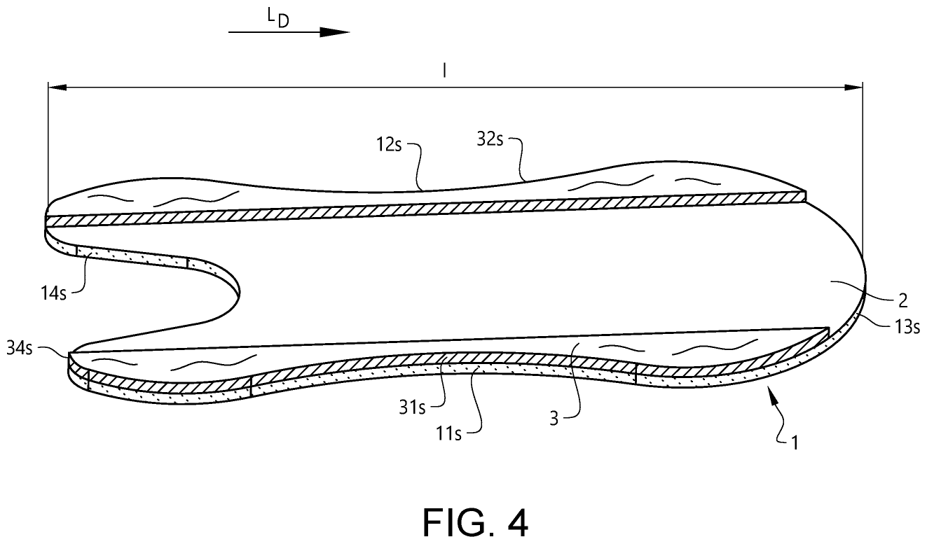

FIG. 4 illustrates a shaped permeable material composite 1 as disclosed herein and for use in an absorbent article. The permeable material composite 1 has a length 1 in a longitudinal direction LD extending between a first shaped transverse edge 13s and a second shaped transverse edge 14s of the permeable material layer 2. The permeable material composite 1 has a width in a transverse direction extending between a first shaped longitudinal edge 11s and a second shaped longitudinal edge 12s of the permeable material layer 2. The first transverse edge 13s is rounded outwardly in a convex shape and the second transverse edge 14s has the shape of two legs extending in a rearwards direction. The first and second longitudinal edges 11s,12s each has a slightly curved shape, such that the permeable material composite 1 has a slightly inwardly curved waist portion. The permeable material layer 2 has a reinforcing material 3 in the form of two longitudinally extending strips attached thereto. The reinforcing material 3 thus has shaped outer first and second longitudinal edges 31s,32s with contours coinciding fully with the contours of the first and second longitudinal edges 11s,12s of the permeable material layer 1.

The permeable material layer 2 is a fibrous layer comprising non-absorbent synthetic fibers, the layer has a tensile strength of less than 3 N/mm. The reinforcing layer 3 is a nonwoven layer extending over the longitudinal edges 11s,12s of the permeable material layer 2 and is formed by two strips of reinforcing material 3 as shown in FIG. 2b.

In the table below there are provided examples of reinforcement material and low-strength material in accordance with the present disclosure.

TABLE 1
(all measurements in N/mm)

| | Lower | Target | Upper |
|---|---|---|---|
| REINFORCEMENT | | | |
| MATERIAL | | | |
| Tissue 17gsm | 0.12 | 0.15 | |
| Tissue 16gsm 100% bleached | 0.18 | 0.28 | |
| ECF or TCF pulp | | | |
| Spunbond NW 18gsm | 0.44 | 0.76 | 1.12 |
| Spunbond NW 16gsm | 0.46 | 0.74 | 1.02 |
| Spunbond NW 22gsm | 0.64 | 0.98 | 1.32 |
| LOW STRENGTH | | | |
| MATERIAL | | | |
| Air Laid 70gsm multibonded | 0.2 | 0.34 | |
| Air Laid 65gsm multibonded | 0.36 | 0.34 | |
| Foam 3M1A | | 0.22 | |
| Foam Recticil T25090 | | 0.17 | |
| Foam Woodbridge SM32 WH | | 0.22 | |

The embodiments described above are only descriptions of preferred embodiments of the present invention, and are not intended to limit the scope of the invention. Various variations and modifications can be made to the technical solution of the present invention by those of ordinary skill in the art, without departing from the design of the present invention. The variations and modifications should all fall within the claimed scope defined by the claims of the invention.

What is claimed is:

1. A permeable material composite (1) comprising a permeable material layer (2) being a low-strength permeable material layer having a tensile strength of less than 3 N/mm for use in an absorbent article, said permeable material layer (2) having an edge (11,12,13,14) having a contour and wherein said edge (11,12,13,14) is a shaped edge (11s,12s,13s,14s) providing the permeable material composite (1) with a non-rectangular shape;
characterized in that said permeable material layer (2) is reinforced along said shaped edge (11s,12s,13s,14s) with a reinforcement material (3) being attached to said permeable material layer (2), said reinforcement material (3) having an outer edge (31s,32s,33s,34s) having a contour, and wherein said contour of said shaped outer edge (31s,32s,33s,34s) of said reinforcement material (3) coincides fully or partly with said contour of said shaped edge (11s,12s,13s,14s) of said permeable material layer (2).

2. The permeable material composite (1) according to claim 1, wherein said permeable material layer (2) has a length (I) in a longitudinal direction (L_{D}) extending between a first and a second transverse edge (13,14) and a width (w) in a transverse direction (T_{D}) extending between a first and a second longitudinal edge (11, 12), said longitudinal edges (11,12) and said transverse edges (13,14) each having a contour and wherein at least one of said edges (11,12,13,14) is said shaped edge (11s,12s,13s,14s).

3. The permeable material composite (1) according to claim 2, wherein said first and said longitudinal edges (11s,12s) are shaped first and second longitudinal edges (11s,12s).

4. The permeable material composite (1) according to any one of claims 1-3, wherein said permeable material layer (2) is a liquid permeable fibrous layer or a liquid permeable foam layer.

5. The permeable material composite (1) according to claim 4, wherein in the fibers in said liquid permeable fibrous layer consist of non-absorbent synthetic fibers.

6. The permeable material composite (1) according to any one of claims 1-5, wherein said permeable material layer (2) comprises superabsorbent particles.

7. The permeable material composite (1) according to any one of claims 1-6, wherein said reinforcing material (3) is a nonwoven material, such as a spunbond nonwoven material, or a tissue material.

8. The permeable material composite (1) according to any one of claims 1-7, wherein said reinforcing material (3) has a tensile strength of 0.1 N/mm or more.

9. The permeable material composite (1) according to any one of the preceding claims, wherein said permeable material layer (2) has a tensile strength of from 0.05 to 3 N/mm.

10. The permeable material composite (1) according to any one of the preceding claims, wherein said shaped edge (11s,12s,13s,14s) of said permeable material layer (2) is/co-shaped with said outer shaped edge (31s,32s,33s,34s) of said reinforcing material (3).

11. The permeable material composite (1) according to any one of the preceding claims, wherein said reinforcing material (3) further comprises an active ingredient, such as an odour control substance, a print, a hydrophobic composition and/or elastic elements.

12. An absorbent article comprising a liquid permeable topsheet and a backsheet, wherein said permeable material composite (1) according to any one of the preceding claims is enclosed between said liquid permeable topsheet and said backsheet.

13. A method for producing a permeable material composite (1,101) having one or more shaped reinforced edge portions for use in an absorbent article, said method comprises the step of;
a) providing a web of a permeable material (200) having a tensile strength of less than 3 N/mm;
b) providing a web of a reinforcing material (300);
c) applying said reinforcing material web (300) over said permeable material web (200) and attaching said reinforcing material web (300) to said permeable material web (200) to provide a permeable material composite web (100) comprising a reinforced overlap region (400); and
d) forming said permeable material composite (101) having one or more shaped reinforced edges (111s,112s,113s,114s) by cutting along a cutting line (15) at least partly arranged within said overlap region (400) and removing trim material (4) from said permeable material composite (1,101).

14. The method according to claim 13, wherein said permeable material web (200) has longitudinal edge portions (201,202) extending in a longitudinal direction (L_{D}) of said permeable material web (200) and wherein step b) involves conveying said permeable material web (200) in a machine direction (MD) corresponding to said longitudinal direction (L_{D}) of said permeable material web (200).

15. The method according to claim 14, wherein step c) said reinforcing material (300) is applied to overlap one or both of said first and said second longitudinal edge portions (201,202), and in step d) said permeable material composite (1,101) is formed by cutting or severing at least partly within said first and/or said second longitudinal edge portions (201,202) and removing said trim material (4) from said permeable material composite web (100) forming one or two shaped reinforced edges.

16. The method according to any one of claims 11-15, wherein said trim material (4) has a width and a minimum width of said trim material (4) is 20 mm or less, such as 12 mm or less, such as 7.5 mm or less.

17. The method according to any one of claims 13-16, wherein said step d) involves attaching said reinforcing material web (300) to said permeable material web (200) by means of gluing or by ultrasound.

18. The method according to any one of claims 13-17, wherein said step d) involves cutting or severing said permeable material composite (101,1) in a non-rectangular shape.

19. The method according to any one of the preceding claims, wherein said reinforcing material (300) has a tensile strength greater than said tensile strength of said web of a low-strength permeable material (200).

20. The method according to any one of claims 13-19, wherein said method further comprises the step of;
d) integrating said permeable material composite (1,101) between a topsheet and a backsheet forming an absorbent article.

21. A method for removing trim material (4) when manufacturing a permeable material composite (1,101) for use in an absorbent article comprising a topsheet, a backsheet and said permeable material composite (1,101) therebetween, said method comprises the steps of;
a) providing a web of a permeable material (200) in a machine direction, said permeable material web (200) having a tensile strength, as measured in said machine direction;
b) applying a web of a reinforcing material (300) over said permeable material web (200) and attaching said reinforcing material web (300) to said permeable material web (200) thereby forming a permeable material composite web (100);
c) cutting, or severing, said permeable material composite web (100) in said machine direction (MD) so as to form a trim material (4), said trim material (4) comprising at least a portion of said permeable material web (200) and a portion of said reinforcing material web (300); and
d) removing said trim material (4) by pulling said trim material (4) with a pulling force, wherein said pulling force is higher than said tensile strength per millimetre of said permeable material web (200).

22. The method according to claim 21, wherein said pulling force in step d) is 1 N or more.

23. The method according to claim 21 or 22, wherein said permeable material web (200) has a tensile strength of less than 3 N/mm.
